(19) United States
(12) Patent Application Publication
(10) Pub. No.: US 2021/0355427 A1
(43) Pub. Date: Nov. 18, 2021

(54) USE OF VISION SYSTEMS IN BIOMANUFACTURING PROCESSES

(71) Applicant: EMD MILLIPORE CORPORATION, Burlington, MA (US)

(72) Inventors: Anne Hansen, North Reading, MA (US); Kevin O'Brien, Lexington, MA (US); Amy Wood, Concord, MA (US); Noah Rhein, Maynard, MA (US); Thomas Janko, Stoneham, MA (US); Oliver Hiller, Röttenbach (DE)

(21) Appl. No.: 17/312,960

(22) PCT Filed: Mar. 26, 2020
(86) PCT No.: PCT/US2020/025040

§ 371 Date: Jun. 11, 2021

Related U.S. Application Data
(60) Provisional application No. 62/825,351, filed on Mar. 28, 2019.

(51) Int. Cl. C12M 1/36; C12M 1/34

(52) U.S. Cl. CPC C12M 41/48; C12M 41/44

(57) ABSTRACT

A system for processing a biological fluid that includes a bioreactor, wherein the bioreactor includes a window, at least one port for allowing delivery of a processing aid; a control system, a sensor; a transmitter for transmitting the signal; a signal converter; a controller for receiving the signal; and a valve for delivering the processing aid to the port, wherein the sensor senses a process condition, transmits the signal, and the signal is compared with a reference signal and wherein a process action is optionally taken based on the comparison.

### RELATED APPLICATIONS

The present application claims the benefit of U.S. Provisional Application No. 62/825351, filed on Mar. 28, 2019, the entire contents of which is incorporated by reference herein in its entirety.

### BACKGROUND

### Field of the Disclosure

This disclosure relates generally to systems for containing, processing and manipulating biological fluids. More specifically, in some embodiments, systems and methods comprising steel bioreactors or flexible, collapsible bags that may be used as reactors for performing biochemical, biological reactions, and/or cell growth, and the like contained therein, are described.

### Description of the Prior Art

Many types of vessels for containing, processing and manipulating biological fluids are available. For example, biological materials, e.g., cells, including, for example, mammalian and plant cells, and viral or microbial cultures, can be cultured using bioreactors. Traditional bioreactors, for e.g., steel vessels, or disposable bioreactors, many of which use plastic bags, may be used. During processing, additives, such as various feedstocks, oxygen, pH buffers and salts, and other processing aids are added to the biological fluid, which contain cell cultures. Furthermore, these additives are mixed using strong impellers and may include the use of baffles to achieve more ideal mixing criteria.

The bioprocessing of the cell cultures must be monitored, either manually or with instrumentation. Various sensors are generally used within such bioreactors and bags to determine the state or condition of the biological liquid or cells within the bag. Such sensors typically monitor pH, dissolved gases, temperature, turbidity, conductivity, biomass, metabolites and/or inhibitors, products of interest and the like to determine homogeneity of such properties throughout the bioreactor or bag. To do so, sensors are often placed within dip tubes from the top of the bag into the inner volume of the bag at one or more locations. Alternatively, sensors are simply mounted to an inner wall of the bioreactor. The use of such sensors can be cost prohibitive. If the sensors are to be re-used, they must be cleaned and sterilized. In some cases, the sensors are single use sensors, which are then discarded.

Because of vigorous mixing of a biological fluid and additives, foam often forms on a surface of the biological fluid being processed, which is unfavorable. Anti-foam additives are added to lessen the amount of foam. However, the addition of the anti-foam additive is dependent upon manual intervention, creating a condition of constant monitoring. And, the amount of anti-foam additive and timing of the addition is both reactive and subjective. A process and instrumentation, such as sensors and cameras, for adding anti-foam additives in a process-controlled, automatic manner represents an advance in the art. Furthermore, an automatic process for monitoring the turbidity, color, and other properties for controlling the status of the biological fluid represents an advance in the art.

### SUMMARY

Embodiments of this disclosure relate to systems and methods for containing, processing, and manipulating biological fluids and, in some embodiments, to systems and methods comprising steel tanks and flexible, collapsible bags that may be used as bioreactors, further comprising fluid level sensors and/or cameras which are disposed outside the bioreactor or bag, substantially as shown in and/or described in connection with at least one of the figures, as set forth more completely in the claims. A system for processing a biological fluid comprising a bioreactor, wherein the bioreactor includes a window, at least one port for allowing delivery of a processing aid; a control system, a sensor; a transmitter for transmitting the signal; a signal converter; a controller for receiving the signal; and a mechanism, such as a valve or a pump, for delivering the processing aid to the port, wherein the sensor senses a process condition, transmits the signal, and compares the signal versus a reference signal, data point, and/or stored reference data, wherein a process action is optionally taken based on the comparison and methods related thereto are disclosed.

Various benefits, aspects, novel and inventive features of the present disclosure, as well as details of exemplary embodiments thereof, will be more fully understood from the following description and drawings.

### BRIEF DESCRIPTION OF THE DRAWINGS

FIG. 1 represents a front view of a steel bioreactor having a window and a control system in communication therewith, according to some embodiments described in the disclosure;

FIG. 2 represents a front view of a flexible bag bioreactor having a window and a sensor, according to some embodiments described in the disclosure;

FIG. 3 is a flow diagram depicting a method for treating a biological fluid, according to some embodiments described in the disclosure;

FIG. 4 is a flow diagram depicting a second method for treating a biological fluid, according to some embodiments described in the disclosure; and

FIG. 5 depicts a bioreactor having an internal volume that contains a region having a liquid, a region having foam, and a region of air, according to embodiments of the disclosure.

### DETAILED DESCRIPTION

So the manner in which the features disclosed herein can be understood in detail, a more particular description of the embodiments of the disclosure, briefly summarized above, may be had by reference to the appended drawings. It is to be noted, however, that the appended drawings illustrate only typical embodiments of the disclosure and are therefore not to be considered limiting of its scope, for the embodiments disclosed herein may admit to other equally effective embodiments. It is also to be understood that elements and features of one embodiment may be found in other embodiments without further recitation and that, where possible, identical reference numerals have been used to indicate comparable elements that are common to the figures.

Any of the bioreactors, bags, or containers described herein may include one or more transparent windows so that the contents, e.g., biological fluids, thereof may be identified by a sensor, for example, a fluid level sensor and/or a camera. Any embodiment of the bioreactor, bag, or container described herein is of a sufficient size to contain a biological fluid, such as cells and a culture medium, to be mixed, from, for e.g., bench-top scale to 3000 L bioreactors.

The fluid level sensors and/or cameras are capable of detecting many conditions. For example, foaming, leaks, volume-level, color, turbidity, clarity, homogeneity, flow, and/or bulging of the bag or a change in shape because of pressure changes.

In accordance with some embodiments, the bioreactor is designed to receive and maintain a liquid or a fluid. In some embodiments, the bioreactor is a stainless-steel bioreactor. In some embodiments, the bioreactor is a flexible, single use bag.

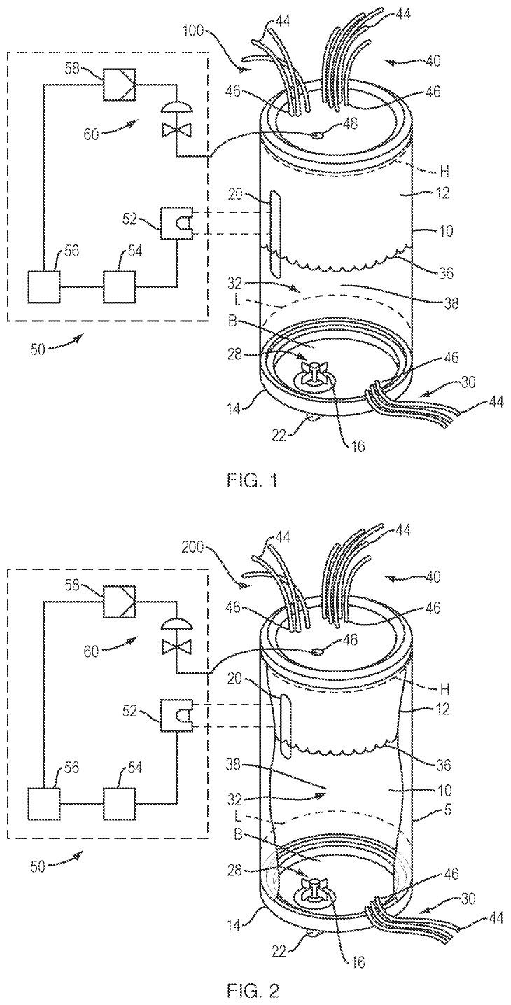

Turning now to the drawings, FIG. 1 represents a front view of a steel bioreactor 100 having a transparent window 20 and a control system 50 in communication therewith, according to some embodiments described in the disclosure. The steel bioreactor 100 generally comprises a wall 10 formed in a cylindrical shape and having an internal working volume 32. The internal working volume 32 is capable of processing liquids of a very small amount, e.g., 0.5 liters (L) to, for example, 4000 L without substantially changing shape.

The control system 50 comprises a sensor 52 for generating a signal, a transmitter 54 for transmitting the signal, a signal converter 56, a controller 58, and a valve 60. The sensor 52, which may be, for e.g., a camera or a fluid level sensor, is capable of sensing the presence and/or height of a foam 36 disposed on a surface 38 of a fluid within the inner working volume 32. Some exemplary sensors and/or image-generating devices are marketed by Cognex Corp., of Natick, Mass., USA, Omron Corp., of Kyoto, Japan, and/or Keyence Corp., of Osaka, Japan. The controller 58 may be a dedicated microprocessor, i.e., a computer. Alternatively, the controller 58 may be a computer, iPad®, or other personal digital assistant that is capable of receiving a signal and providing instructions to the output mechanism and being controlled from a remote location. The output mechanism may be a pump or a valve. The valve 60 may be any style valve capable of receiving a signal for opening and dosing. In some systems, the input of the various “processing aids,” e.g., anti-foam additives, are controlled by a metering pump, such as a peristaltic pump, which, optionally, is in communication with the controller 58. Such valve(s) comprise a pneumatic, a hydraulic, or an electrical valve. It is to be understood that the control system 50 is capable of providing real-time feedback and control, i.e., a servo control, Proportional-Integral-Derivative (PID) control, and the like. For example, the signal generated by the sensor 52 is capable of instructing the valve 60 to deliver an agent or processing aid, such as an anti-foam additive. Furthermore, the control system 50 is capable, of instructing the valve 60 to deliver differing or varied amounts of an agent or processing aid based on, for example, the height of the foam 36 detected on the surface 38 of the fluid being processed. The agent or processing may be added into the inner working volume 32 via 48 or via inlet 44.

The bioreactor 100 has an impeller assembly 28, further comprising a base 14 and one or more moveable blades or vanes 16. In some embodiments, the driver, such as a motor (not shown) for the impeller assembly 28, is external to the bioreactor 100. In some embodiments, the container 10 has a minimum internal working volume of 0.5 L, and a maximum internal working volume of 4000 L. It is to be understood that, irrespective of size, the bioreactor 100 need not be at full liquid capacity to operate. For example, any bioreactor 100, whether 200 L or 3000 L may operate at a maximum internal working volume H or, alternatively, a minimum internal working volume L, which is at a liquid height just above the impeller assembly 28. The bioreactor 100 may also operate at any working internal volume between the maximum working volume H and the minimum working volume L. In some embodiments, at least a portion of the impeller assembly 28 is disposed within the internal working volume 32 of the bioreactor 100.

The number and shape of the blades 16 of the impeller assembly 28 is not particularly limited, provided the blades 16 are capable of sufficiently agitating a fluid within the bioreactor 100 when actuated. The blades may be constructed of plastic material, such as polyethylene, or any polymer resistant to gamma irradiation, such as polypropylene or a polypropylene co-polymer, for sterilization purposes. The bioreactor 100 optionally comprises wherein the base 14 is constructed of plastic material, such as polyethylene, or any polymer resistant to gamma irradiation, such as polypropylene, or a polypropylene co-polymer, also for sterilization purposes. The bioreactor 100 may have a relatively flat bottom B or, alternatively, a conical bottom (not shown) or other tapered bottom. The bioreactor 100 may, alternatively, comprise a two-dimensional tapered bottom (not shown).

In some embodiments, the base 14 includes an axially extending member 22. The axially extending member 22 accommodates a magnetic base of the impeller assembly 28, such as a mixing impeller overmolded magnet (not shown), wherein the blades 16 extend axially above the member 22 and are free to rotate when the magnetic impeller is driven by a drive magnet. In some embodiments, wherein the impeller assembly 28 is installed in the bioreactor 100, the extending member 22 protrudes outside the bioreactor 100, wherein the base 14 is sealed to the bioreactor 100. The remainder of the impeller assembly 28 is housed inside the bioreactor 100. In some embodiments, the impeller assembly 28 is placed at or near the bottom B of the bioreactor 100, wherein the bioreactor 100 is in mixing position (such as a hanging position) and proximal to at least one port 46, such outlet(s) 30 of the bioreactor 100.

The bioreactor 100 further comprises a plurality of baffle inlets 40. Fluid access into the inner working volume 32 is via one or more of a plurality of ports 46. The plurality of ports 46 are, optionally, adhered, connected, sealed, or otherwise welded directly to the bioreactor 100. Each or any of the plurality of ports 46 may comprise a plug (not shown), a connector (not shown) or have a conduit or tube 44 attached or formed integrally therewith. In some exemplary embodiments, the tube(s) 44 are formed of a silicone material, which is suitable of sterilization via radiation. In some exemplary embodiments, the tube(s) 44 are formed of weldable tubing material. It is further noted that fluid can exit the bioreactor via ports 30. For example, the bioreactor 100 comprises a plurality of exit ports 30 proximal the Bottom B of the bioreactor 100.

In some embodiments, the exit ports 30, and/or the plurality of inlet baffle inlets 40 comprise a one-way valve (not shown) or a hydrophobic membrane (not shown) so that liquid (with the valve) or gas (with the valve or hydrophobic membrane) can only selectively enter or exit therethrough, as may be desired.

FIG. 2 represents a front view of a flexible bioreactor bag 200 having a plastic window 22 and a sensor 50, according to some embodiments described in the disclosure. The flexible bioreactor bag 200, which may be a single-use bioreactor, generally comprises a wall 12 formed in a generally cylindrical shape and having an internal working volume 32. The flexible bioreactor bag 200 may be housed in, for example, a shell 5. The internal working volume 32 is capable of processing liquids of a very small amount, e.g., 0.5 L to, for example, 4000 L without substantially changing shape.

As above, the control system 50 comprises a sensor 52 for generating a signal, a transmitter 54 for transmitting the signal, a signal converter 56, a controller 58, and a valve 60. The sensor 52, which may be, for e.g., a camera or a fluid level sensor, is capable of sensing the presence and/or height of a foam 36 disposed on a surface 38 of a fluid within the inner working volume 32 via the window 22. As above, a camera or fluid sensor may be supplied by any of various manufacturers as are known to those in the art. The controller 58 may be a dedicated microprocessor, i.e., a computer. Alternatively, the controller 58 may be a computer, a local process automation control skid, a centralized process automation control skid, an iPad, or other personal digital assistant that is capable of receiving a signal and providing instructions to the valve 60 and being controlled from a remote location. The valve 60, or metering system, as described above, may be any style valve capable of receiving a signal for opening and closing. Such valve(s) comprise, a pneumatic, a hydraulic, or an electrical valve. It is to be understood that the control system 50 is capable of providing real-time feedback and control, i.e., a servo control, Proportional-Integral-Derivative (PID) control, and the like. For example, the signal generated by the sensor 52 is capable of instructing the valve 60 to deliver an agent or processing aid, such as an anti-foam additive. And, the signal generated by the sensor 52 is capable of instructing the valve 60 to deliver an agent or processing aid, such as an anti-foam additive. Furthermore, the control system 50 is capable of instructing the valve 60 to deliver differing or varied amounts of an agent or processing aid based on, for example, the height of the foam 36 detected on the surface 38 of the fluid being processed. The agent or processing may be added into the inner working volume 32 via 48 or via inlet 44.

The flexible bioreactor bag 200 has an impeller assembly 28, further comprising a base 14 and one or more moveable blades or vanes 16. In some embodiments, the driver, such as a motor (not shown) for the impeller assembly 28, is external to the flexible bioreactor bag 200. In some embodiments, the flexible bioreactor bag has a minimum internal working volume of, for e.g., 0.5 L-10L, and a maximum internal working volume of 4000 L. It is to be understood that, irrespective of size, the flexible bioreactor bag 200 need not be at full liquid capacity to operate. For example, any flexible bioreactor bag 200, whether, e.g., 10 L or 4000 L may operate at a maximum internal working volume H or, alternatively, a minimum internal working volume L, which is at a liquid height just above the impeller assembly 28. The flexible bioreactor bag 200 may also operate at any working internal volume between the maximum working volume H and the minimum working volume L. In some embodiments, at least a portion of the impeller assembly 28 is disposed within the internal working volume 32 of the flexible bioreactor bag 200.

The number and shape of the blades 16 of the impeller assembly 28 is not particularly limited, provided the blades 16 are capable of sufficiently agitating a fluid within the flexible bioreactor bag 200 when actuated. The blades may be constructed of plastic material, such as polyethylene, or any polymer resistant to gamma irradiation, such as polypropylene or a polypropylene co-polymer, for sterilization purposes. The flexible bioreactor bag 200 optionally comprises wherein the base 14 is constructed of plastic material, such as polyethylene, or any polymer resistant to gamma irradiation, such as polypropylene, or a polypropylene co-polymer, also for sterilization purposes. The flexible bioreactor bag 200 may have a relatively flat bottom B or, alternatively, a conical bottom (not shown) or other tapered bottom. The flexible bioreactor bag 200 may, alternatively, comprise a two-dimensional tapered bottom (not shown).

In some embodiments, the base 14 includes an axially extending member 22. The axially extending member 22 accommodates a magnetic base of the impeller assembly 28, such as a mixing impeller overmolded magnet (not shown), wherein the blades 16 extend axially above the member 22 and are free to rotate when the magnetic impeller is driven by a drive magnet. In some embodiments, wherein the impeller assembly 28 is installed in the bioreactor 100, the extending member 22 protrudes outside the flexible bioreactor bag 200, wherein the base 14 is sealed to the flexible bioreactor bag 200. The remainder of the impeller assembly 28 is housed inside the flexible bioreactor bag 200. In some embodiments, the impeller assembly 28 is placed at or near the bottom B of the flexible bioreactor bag 200, wherein the flexible bioreactor bag 200 is in a mixing position (such as a hanging position) and proximal to at least one port 46, such outlet(s) 30 of the flexible bioreactor bag 200.

The flexible bioreactor bag 200, as with the bioreactor 100 described in FIG. 1, further comprises a plurality of baffle inlets 40. Fluid access into the inner working volume 32 is via one or more of a plurality of ports 46. The plurality of ports 46 are, optionally, adhered, sealed, or otherwise welded directly to the flexible bioreactor bag 200. Each or any of the plurality of ports 46 may comprise a plug (not shown) or have a conduit or tube 44 attached or formed integrally therewith. In some exemplary embodiments, the tube(s) 44 are formed of a silicone material, which is suitable of sterilization via radiation. It is further noted that fluid can exit the bioreactor via ports 30. For example, the flexible bioreactor bag 200 comprises a plurality of exit ports 30 proximal the Bottom B of the flexible bioreactor bag 200.

In some embodiments, the exit ports 30, and/or the plurality of inlet baffle inlets 40 comprise a one-way valve (not shown) or a hydrophobic membrane (not shown) so that liquid (with the valve) or gas (with the valve or hydrophobic membrane) can only selectively enter or exit therethrough, as may be desired.

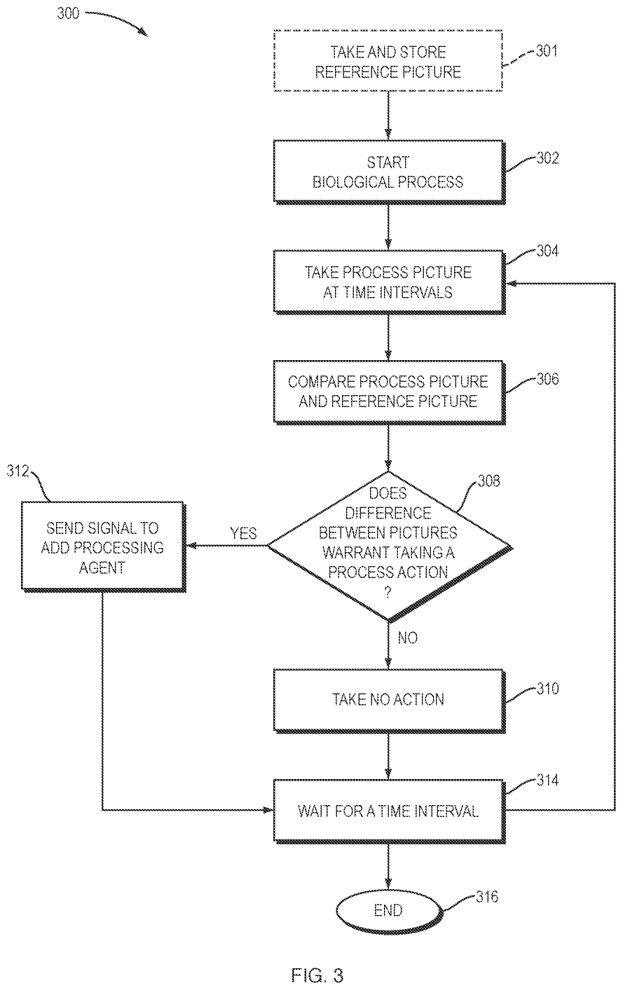

FIG. 3 is a flow diagram depicting a method 300 for treating a biological fluid, according to some embodiments described in the disclosure. A biological process can include, for example, cell culturing, clarification, purification, viral clearance, viral inactivation, polishing, and other biological processes as are known to those in the art. The method 300 optionally comprises taking and storing a reference picture or image at step 301. The image is of a fluid level that requires that no process action be taken. The image may also be of a fluid having no or little foam on a surface of the fluid level indicating that no process action should be taken. For example, a process action can comprise the sending of a signal to a personal digital assistant, i.e., a smart phone, a tablet, an iPad®, or any hand-held microprocessor. The signal may comprise a simple alert. Alternatively, the signal may be part of a feedback loop in which, ultimately, a process action is taken automatically, for example, the opening of a valve to deliver a processing aid or agent into a bioreactor. In some embodiments, the aid or agent comprises a buffer, a salt solution, an anti-foam additive, a pH buffer, a feedstock, nutrients, cell culture media, and/or other additives associated with the processing of biological fluids, cell culture processes, etc.

At step 302, a biological process on a biological fluid is started, for example, a cell culturing process. At step 304, a sensor measures a property of the biological fluid. For example, a fluid level sensor may measure a height of the biological fluid and/or whether a presence of foam is on a surface of the biological fluid. In some embodiments, the sensor comprises a camera. The camera may take a snapshot of the fluid level of the biological fluid and/or foam.

At step 306, a microprocessor or other digital device compares the measured property with a standard. For example, a process picture taken with a camera may be compared with a reference picture.

At step 308, software loaded onto the microprocessor compares the reference picture with the process picture. Also, in some embodiments, the vision system (and software) does not explicitly compare process pictures to reference pictures. Rather, in some embodiments, the vision system performs measurements on the process picture or image and compares those measurements to a reference value. For example, if an acceptable foam level, i.e., one requiring no process action to be taken, is 1.25 centimeters (cm), the alarm/action would be triggered if the process picture was measured and found to have a foam level of, for e.g., 1.3 cm. If the difference between the process picture and the reference picture (or reference value) demonstrates that a process action is taken at step 312. For example, a process action can be sending a signal to a personal digital assistant to someone associated with the process, e.g., a worker. Also, a process action can comprise sending a signal to, for example, energize a valve so that a process aid or agent is delivered into a bioreactor holding and/or processing the biological fluid. A fluid level sensor may also send such a signal. In either of these instances, at step 312, an aid or agent is delivered. If the difference between the reference picture and the process picture are moderate such that no action need be taken, no action is taken at step 310. As above, a fluid level sensor is also capable of making such a determination. In either case, the method 300 proceeds to step 314. A time interval, for example, 1-5 minutes, is allowed to elapse. The method 300 then returns to step 304. This loop continues until, for e.g., the end of the processing of the biological fluid, whereupon the method 300 ends at step 316.

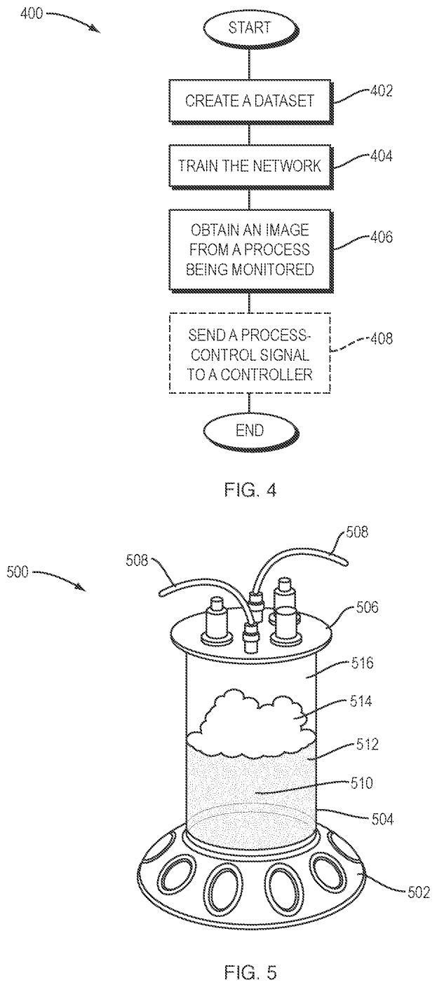

FIG. 4 is a flow diagram depicting a second method 400 for treating a biological fluid, according to some embodiments described in the disclosure. The process 400 starts at step 402, at which point a reference data set is created. The reference data set may comprise, for example, a series or sequence of images or a video, or a series or sequence of images culled from the video. The reference data set can be stored on digital memory, a digital server or any microprocessor having memory. The series or sequence of images or video are made from a biological process within a bioreactor. The images are labeled or classified with respect to different regions within the image, resulting in a labeled image called a mask. One such mask comprises four classes, 1) air, 2) liquid, 3) foam, and 4) an optional background, i.e., everything within the image that is not air, liquid, or foam.

At step 404, a network is trained. An image from the dataset is provided to the network as an input and a prediction is generated. The prediction and the mask image (also called ground truth) are compared and the error or deviation is back propagated through the network. The network then adjusts its parameters to improve its results and to minimize the error or deviation. This adjustment step continues until the network has analyzed and determined what features to look for to make suitable predictions for a model.

At step 406, previously unseen data, e.g., a novel image obtained from a process being monitored, is created. The novel image, from the monitored process, can then be compared with the previously created model and an inference on the new data is made for real-time use and analysis.

At step 408, if a previously determined parameter from the monitored process, e.g., the amount of foam, reaches a threshold for action, an action is optionally taken. The action can be a visual and/or audio alarm. In some embodiments, the action is to send a signal to an instrument in communication with the bioreactor, i.e., an additive, such as an anti-foam additive, is dispensed within the bioreactor at a rate and/or in an amount appropriate to the amount of foam determined in step 406. The process 400 then ends.

It is to be understood that the method 400 comprises a pixel-wise classification, which allows a detection content of the bioreactor (i.e., foam level or height), and also determines the volume of the content by counting pixels. Furthermore, the method 400 can be employed for detecting when the content is fully mixed. For example, the method 400 can be used to automatically determine various powder mixing steps in a biological process and whether the powder is fully mixed, as opposed to requiring an operator's action following a visual inspection.

FIG. 5 depicts a bioreactor 500 having an internal volume that contains a region having a liquid, a region having foam, and a region of air, according to embodiments of the disclosure. The bioreactor 500 comprises a base 502, a cylinder 504, a top 506, and inputs 508. Within the cylinder 504 is an internal volume 510. Shown is the internal volume 510 having a volume of liquid 512, such as a biological fluid, contained therein. Above the liquid 512 is a region of foam 514 and above the region of foam 514 is a region having air 516. Images can be taken of the regions of liquid 512, foam 514, and air 516 to create a model and a mask and a monitored process, as described in the method 400. Any of the sensors, cameras, and other image obtaining devices used as in FIG. 1 can also be incorporated within FIG. 5 without further recitation.

In some embodiments, the flexible bioreactor bag 200 comprises monolayer walls or multilayer flexible walls formed of a polymeric composition such as polyethylene, including ultrahigh molecular weight polyethylene, very low density polyethylene, ultralow density polyethylene, linear low density polyethylene, low density or medium density polyethylene; polypropylene; ethylene vinyl alcohol (EVOH); polyvinyl chloride (PVC); polyvinyl acetate (PVA); ethylene vinyl acetate copolymers (EVA copolymers); thermoplastic elastomers (TPE), and/or blends or alloys of any of the foregoing materials as well as other various thermoplastics materials and additives known to those in the art. The single use bag, owing to the materials from which it is manufactured, is collapsible and expandable. The single use bag may be formed by various processes including, but not limited to, co-extrusion of similar or different thermoplastics; multilayered laminates of different thermoplastics; welding and/or heat treatments, heat staking, calendaring, or the like. Any of the foregoing processes may further comprise layers of woven or non-woven substrates, adhesives, tie layers, primers, surface treatments, and/or the like to promote adhesion between adjacent layers. By “different,” it is meant different polymer types such as polyethylene layers with one or more layers of EVOH as well as the same polymer type but of different characteristics such as molecular weight, linear or branched polymer, fillers and the like, are contemplated herein. Typically, medical grade polymers and, in some embodiments, animal-free plastics are used to manufacture the bags. Medical grade polymers may be sterilized, for e.g., by steam, ethylene oxide or radiation, including beta and/or gamma radiation. Also, most medical grade polymers are specified for good tensile strength and low gas transfer. In some embodiments, the polymeric material is clear or translucent, allowing visual monitoring of the contents and, typically, are weldable and unsupported. In some embodiments, the bag may be a bioreactor capable of supporting a biologically active environment, such as one capable of growing cells in the context of cell cultures. In some embodiments, the bag may be a two-dimensional, i.e., a “pillow” bag or, alternatively, the bag may be a three-dimensional bag. The particular geometry of the bag is not limited in any embodiment disclosed herein. In some embodiments, the bag may include a rigid base, which can provide access points such as ports or vents. Any bag described herein may further comprise one or more inlets, one or more outlets and, optionally, other features such as sterile gas vents, spargers, and ports for the sensing of the liquid within the bag for parameters such as conductivity, turbidity, pH, temperature, dissolved gases, e.g., oxygen and carbon dioxide, and the like as known to those in the art.

In one aspect of some embodiments of the disclosure, the bag may comprise a magnetically-driven antifoaming device, at least a portion of which is positioned in a head space of the bag above a volume of liquid, i.e., biological fluid. The antifoaming device is configured and arranged to break up foam in the head space during rotation of at least a portion of the antifoaming device.

In some embodiments, the bag also comprises a pressure sensor for determining a pressure in the bag, the pressure sensor in fluid communication with the bag, and an antifoarning device associated with the bag and configured to break up foam in the collapsible bag. The bag may also be in communication with a control system operatively associated with the pressure sensor and/or the antifoarning device, wherein the control system regulates the antifoaming device upon receipt of a signal from the pressure sensor.

Systems for containing and manipulating fluids including systems and methods involving supported bags that may be used as reactors for performing chemical, biochemical and/or biological reactions contained therein are provided. Generally, a series of improvements and features for fluid containment systems such as gas delivery configurations, foam control systems and bag molding methods and articles for bioreactors are provided. In some embodiments, fluids contained within a bag can be sparged, e.g., such that a fluid is directed into an inner volume bag, and in some cases, the sparging can be controlled by activating or altering the degree of sparging as needed. Multiple spargers may be used in some cases. In some embodiments, the bag comprises a device which can mechanically reduce the foam produced or contained within the vessel. Sensors and/or controllers may optionally be used to monitor and/or control foaming.

All ranges for formulations recited herein include ranges therebetween and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4, or 3.1 or more.

Reference throughout this specification to “one embodiment,” “certain embodiments,” “one or more embodiments,” “some embodiments,” or “an embodiment” indicates that a feature, structure, material, or characteristic described in connection with the embodiment is included in at least one embodiment of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment.

Although some embodiments have been discussed above, other implementations and applications are also within the scope of the following claims. Although the specification describes, with reference to particular embodiments, it is to be understood that these embodiments are merely illustrative of the principles and applications of the present disclosure. It is therefore to be further understood that numerous modifications may be made to the illustrative embodiments and that other arrangements and patterns may be devised without departing from the spirit and scope of the embodiments according to the disclosure. Furthermore, particular features, structures, materials, or characteristics may be combined in any suitable manner in any one or more of the embodiments.

Publications of patent applications and patents and other non-patent references, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. A system for processing a biological fluid, comprising:
a bioreactor, wherein the bioreactor includes a window;
at least one port for allowing delivery of a processing aid;
a control system, comprising:
a sensor;
a transmitter for transmitting the signal;
a signal converter;
a controller for receiving the signal; and
a valve or mechanism for delivering the processing aid to the port,
wherein the sensor senses a process condition, transmits the signal, and the signal is compared with a reference signal and wherein a process action is optionally taken based on the comparison.

2. The system of claim 1, wherein the sensor comprises a camera or a fluid level sensor.

3. The system of claim 1, wherein the controller comprises a microprocessor.

4. The system of claim 3, wherein the microprocessor is an iPad, a smart phone, an iPhone, a tablet, personal digital assistant, or a hand-held microprocessor

5. A method for treating a biological fluid within a bioreactor, comprising:
optionally comprises taking and storing a reference picture or image of a fluid level and/or foam level disposed on a surface of the fluid;
commencing a biological process on a biological fluid;
generating a signal using a sensor;
sending the signal to a controller;
interrogating a database containing a reference file or standard;
comparing the reference file or standard with the signal; and sending automatically a signal to at least one of a local process automation control skid, centralized process automation control skid, a personal digital assistant, a smart phone, an iPhone, a tablet, an iPad, or a hand-held microprocessor, wherein the signal comprises an alert or a set of process instructions.

6. The method of claim 5, wherein the set of process instructions includes instructions to energize a mechanism or open a valve for delivering a processing aid or agent into the bioreactor.

7. The method of claim 5, wherein the signal senses a fluid level or the presence of a foam.

8. The method of claim 6, wherein the processing aid or agent comprises a buffer, a salt solution, an anti-foam additive, a pH buffer, a feedstock, nutrients, cell culture media, and/or other additives associated with the processing of biological fluids and/or cell culture processes.

9. The method of claim 5, wherein the generating a signal using a sensor step is repeated at known time intervals.

10. The method of claim 9, wherein the time interval is approximately 1 to 5 minutes.

11. A method for treating a biological fluid within a bioreactor, comprising:
commencing a biological process on a biological fluid;
generating a signal using a sensor, wherein the signal is a taking of a process picture or image of a fluid level and/or foam level disposed on a surface of a fluid;
sending the signal to a controller;
comparing the signal with a reference value;
sending automatically a signal to at least one of a local process automation control skid, a centralized process automation control skid personal digital assistant, a smart phone, an iPhone, a tablet, an iPad, or a hand-held microprocessor, wherein the signal comprises an alert or a set of process instructions.

12. The method of claim 11, wherein the set of process instructions includes instructions to open a valve or to energize a metering pump to deliver a processing aid or agent into the bioreactor.

13. The method of claim 12, wherein the processing aid or agent comprises a buffer, a salt solution, an anti-foam additive, a pH buffer, a feedstock, nutrients, cell culture media, and/or other additives associated with the processing of biological fluids and/or cell culture processes.

14. The method of claim 11, wherein the generating a signal step is repeated at known time intervals.

15. The method of claim 14, wherein the time interval is approximately 1 to 5 minutes.

16. A method for treating a biological fluid within a bioreactor, comprising:
creating a reference data set;
training a network;
obtaining one or more images from a biological process being monitored during processing;
comparing the one or more images with the reference data set; and
send a signal to an instrument in communication with the bioreactor.

17. The method for treating a biological fluid within a bioreactor of claim 16, wherein the reference data set may comprise, for example, a series or sequence of images or a video, or a series or sequence of images culled from the video.

18. The method for treating a biological fluid within a bioreactor of claim 17, wherein the reference data set is stored on digital memory, a digital server or any microprocessor having memory.

19. The method for treating a biological fluid within a bioreactor of claim 16, wherein the images are labeled or classified with respect to different regions within the image.

20. The method for treating a biological fluid within a bioreactor of claim 19, wherein labels include 1) air, 2) liquid, 3) foam, and 4) or a background.

21. The method for treating a biological fluid within a bioreactor of claim 16, wherein the network adjusts parameters to improve results.

22. The method for treating a biological fluid within a bioreactor of claim 16, wherein the one or more images obtained from a process being monitored, is made for real-time use and analysis.

23. The method for treating a biological fluid within a bioreactor of claim 16, wherein the signal is a visual and/or audio alarm.

24. The method for treating a biological fluid within a bioreactor of claim 16, wherein a signal is to an instrument in communication with the bioreactor, dispensing an anti-foam additive in an amount appropriate to an amount of foam determined in the obtaining one or more images step.

25. The method for treating a biological fluid within a bioreactor of claim 16, wherein the obtaining one or more images from a biological process being monitored comprises a pixel-wise classification.
